(19) United States
(12) Patent Application Publication
(10) Pub. No.: US 2025/0318793 A1
(43) Pub. Date: Oct. 16, 2025

(54) C-ARM X-RAY IMAGING DEVICES WITH REPOSITIONABLE X-RAY SOURCE OR DETECTOR

(71) Applicant: TURNER IMAGING SYSTEMS, INC., OREM, UT (US)

(72) Inventors: Michael P. ORTHNER, Bountiful, UT (US); Thomas L. YOUD, Holladay, UT (US)

(21) Appl. No.: 18/634,363

(22) Filed: Apr. 12, 2024

(51) Int. Cl. A61B 6/00

(52) U.S. Cl. CPC A61B 6/4441; A61B 6/4405; A61B 6/4411; A61B 6/4452

(57) ABSTRACT

Repositionable C-arm X-ray devices that are portable or mobile and allow the source and/or the detector to be moved from an imaging position to a position outside the field of a medical procedure are described. In particular, this application describes a C-shaped support arm, an X-ray source contained in a first portion of the support arm near a first end, an X-ray detector contained in a second portion of the support arm near a second end, wherein the C-shaped support arm contains a repositioning mechanism between the first and second portions that allows the location of the X-ray source and/or the X-ray detector to be repositioned relative to each other. This configuration allows an X-ray image to be taken of a patient, then the X-ray source and/or detector can be moved outside the sterile field or any location where a medical procedure is performed. Other embodiments are described.

### FIELD

This application relates generally to X-ray equipment. More specifically, this application relates to repositionable C-arm X-ray devices that are handheld, portable, or mobile and that allow the source and/or the detector to be moved from an imaging position to a position outside the field of a medical procedure that is performed on patient.

### BACKGROUND

X-ray imaging systems typically contain an X-ray source and an X-ray detector. The X-ray detector is often an image intensifier or even a flat panel digital detector. X-ray imaging systems are often configured as a C-arm assembly with the source and detector on opposite ends of the “C” arm. The C-arm assembly can move through continuous translational, orbital, and/or rotation angles relative to the object in order to acquire images from multiple orientations.

Medical X-ray imaging equipment, especially when configured as a C-arm, is traditionally heavy and cumbersome to move and position in order to obtain the desired patient images within the operating room environment. Because the equipment is heavy, and draws significant electrical power, it has not been possible to make the devices light enough and small enough to be portable, hand-carried, and/or hand-operated. Advances in many technologies associated with X-ray systems are changing this situation and opening up new applications and much greater ease of use than before.

### SUMMARY

This application relates generally to repositionable C-arm X-ray devices that are portable or mobile and that allow the X-ray source and/or detector to be moved from an imaging position to a position outside the field of a medical procedure, or even just outside the sterile field that might be required for a medical procedure. In particular, this application describes X-ray systems and devices containing a C-shaped support arm, an X-ray source contained in a first portion of the support arm near a first end, and an X-ray detector contained in a second portion of the support arm near a second end, wherein the C-shaped support arm contains a repositioning mechanism between the first and second portions that allows the location of the X-ray source and/or the X-ray detector to be repositioned relative to each other. This configuration allows an X-ray image to be taken of a patient and then the X-ray source and/or detector can be moved outside the location of where a medical procedure needs to be performed.

### BRIEF DESCRIPTION OF THE DRAWINGS

The following description can be better understood in light of the Figures which show various embodiments and configurations of the X-ray systems and devices.

FIG. 1 shows a view of some embodiments of portable and mobile X-ray devices;

FIG. 2 shows other embodiments of the portable and mobile X-ray devices;

FIG. 3 shows yet other embodiments of portable and mobile X-ray devices;

FIG. 4 shows some embodiments of repositionable X-ray devices in an imaging position;

FIG. 5 shows the embodiments of FIG. 4 in an expanded position;

FIG. 6 shows other embodiments of repositionable X-ray devices in an imaging position;

FIG. 7 shows the embodiments of FIG. 6 in an expanded position;

FIG. 8 shows yet other embodiments of repositionable X-ray devices in an imaging position;

FIG. 9 shows the embodiments of FIG. 8 in a rotated position;

FIG. 10 shows even other embodiments of repositionable X-ray devices in an imaging position; and

FIG. 11 shows the embodiments of FIG. 10 in a position slid away from the imaging position.

Together with the following description, the Figures demonstrate and explain the principles of the structures, methods, and principles described herein. In the drawings, the thickness and size of components may be exaggerated or otherwise modified for clarity. The same reference numerals in different drawings represent the same element, and thus their descriptions will not be repeated. Furthermore, well-known structures, materials, or operations are not shown or described in detail to avoid obscuring aspects of the described devices.

### DETAILED DESCRIPTION

The following description supplies specific details in order to provide a thorough understanding. Nevertheless, the skilled artisan will understand that the described X-ray devices can be implemented and used without employing these specific details. Indeed, the described systems and methods for controlling X-ray devices can be placed into practice by modifying the described systems and methods and can be used in conjunction with any other apparatus and techniques conventionally used in the industry. For example, while the description below focuses on C-arm X-ray systems and devices, other X-ray imaging arms and X-ray devices can be used, including U-arms or portable X-ray devices with separate detectors that are configured to approximate a C-arm configuration, including any X-ray system with a fixed source or detector gantry where the imaging function impedes access to the patient.

In addition, as the terms on, disposed on, attached to, connected to, or coupled to, etc. are used herein, one object (e.g., a material, element, structure, member, etc.) can be on, disposed on, attached to, connected to, or coupled to another object-regardless of whether the one object is directly on, attached, connected, or coupled to the other object or whether there are one or more intervening objects between the one object and the other object. Also, directions (e.g., on top of, below, above, top, bottom, side, up, down, under, over, upper, lower, lateral, orbital, horizontal, etc.), if provided, are relative and provided solely by way of example and for ease of illustration and discussion and not by way of limitation. Where reference is made to a list of elements (e.g., elements a, b, c), such reference is intended to include any one of the listed elements by itself, any combination of less than all of the listed elements, and/or a combination of all of the listed elements. Furthermore, as used herein, the terms a, an, and one may each be interchangeable with the terms at least one and one or more.

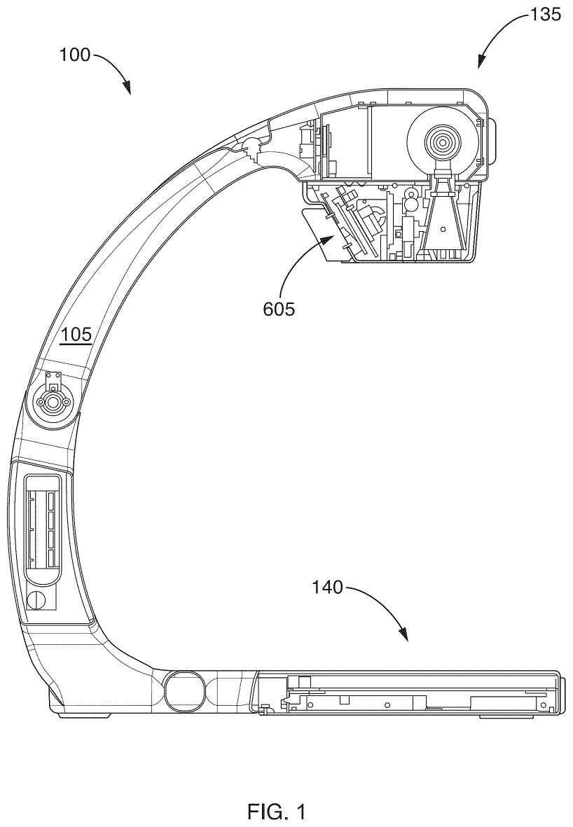

FIGS. 1-11 show some embodiments of the repositionable C-arm X-ray devices that are handheld, portable, or mobile and that allow the source and/or the detector to be moved from the imaging position to a position outside the field of a medical procedure (or outside a sterile field) that needs to be performed on a patient. As shown in FIG. 1, the repositionable X-ray devices 100 contain an imaging arm (or support arm) that allows the X-ray system to be used to take X-ray images of a portion of a patient's body or any other object capable of being analyzed by X-rays, including animals, industrial components such as electronic circuit boards, containers to be inspected, and/or passenger luggage. In some configurations, the imaging arm is substantially shaped like the letter “C” and is therefore referred to as a C-shaped support arm (or C-arm) 105. The C-arm has any size that can be held and operated by hand when in use, as seen in FIG. 1, or any size that is portable or mobile and can be used in combination with any desired medical procedure. In some configurations, the X-ray devices 100 can be handheld so they can be operated by hand. In other configurations, the X-ray devices 100 can be portable since they can be carried by hand. In other configurations, they can be attached, connected, or mounted to a movable external structure so that they are mobile. In yet other configurations, the X-ray devices include any motor-driven C-arms that can used for 3D imaging.

The C-arm 105 can contain any X-ray source 135 and X-ray detector 140 that allow the X-ray device 100 to take X-ray images. The X-ray source 135 can contain any source that generates and emits X-rays, including a standard stationary anode X-ray source, a micro-focus X-ray source, a rotating anode X-ray source, and/or a fluoroscopic X-ray source. In some embodiments, such as with some portable systems, the X-ray source 135 can operate with about 40 kV to about 90 kV and from about 1 to about 10 mA. In other embodiments, such as with some mobile systems, the X-ray source can operate with about 40 KV to about 120 KV and from about 1 mA to about 250 mA. In still other embodiments, such as with some handheld units, the X-ray source can operate with about 75 kV and about 2 mA. In some configurations, the X-ray source and X-ray detector can be made modular so that different sizes and types of X-ray sources and X-ray detectors can be used in the X-ray device 100.

The X-ray detector 140 can comprise any detector that detects X-rays, including an image intensifier, a CMOS camera, a digital flat panel detector, and/or digital direct conversion detectors. In some configurations, the detector can have a substantially square shape with a side ranging from about 10 cm to about 20 cm. In other configurations, though, the X-ray detector 140 does not need to have a substantially square shape. In addition, the X-ray detector 140 can have a pixel size that ranges from a square with dimensions ranging from about 25 μm to about 200 μm to a rectangle with the two dimensions of the rectangular pixel fitting in about the same range. In some configurations, the detector can have a substantially square shape with a length of one side ranging from about 15 cm to about 43 cm. In other configurations, the detector can have a substantially rectangular shape with the shorter dimension ranging from about 20 cm to about 30 cm and the longer dimension ranging from about 30 cm to about 45 cm.

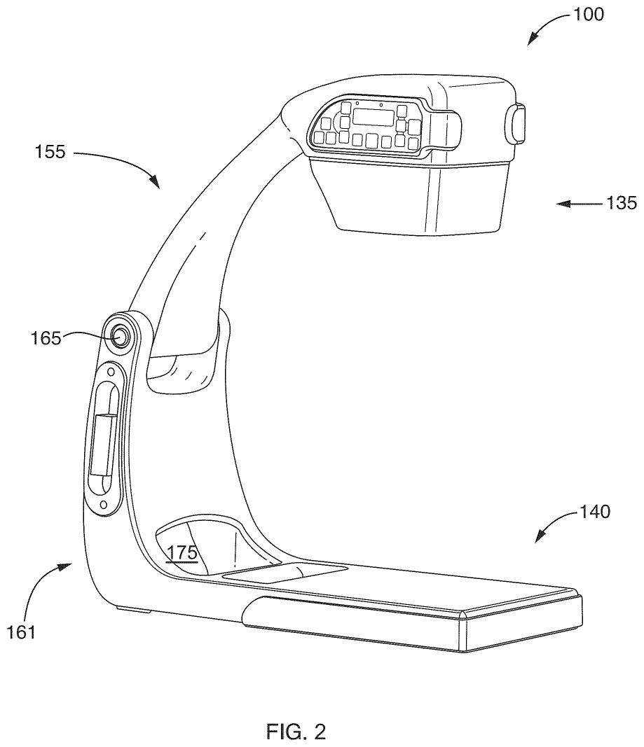

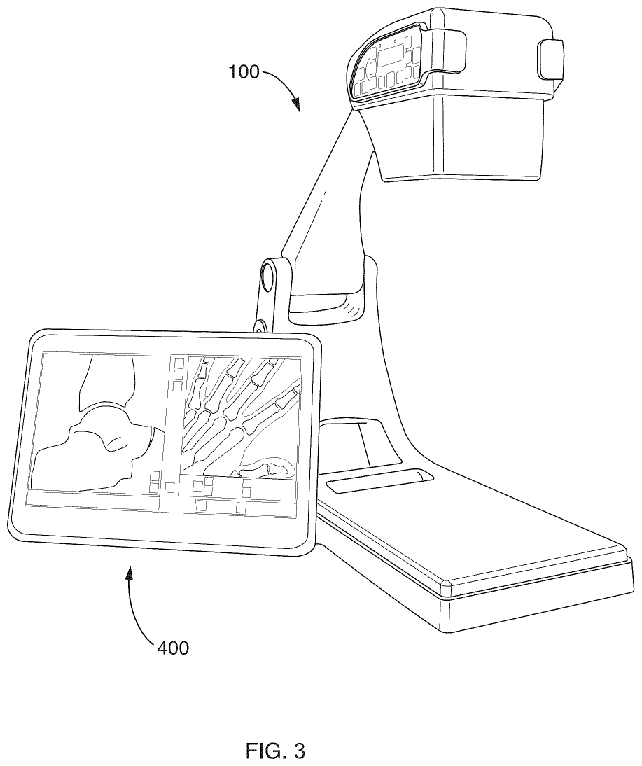

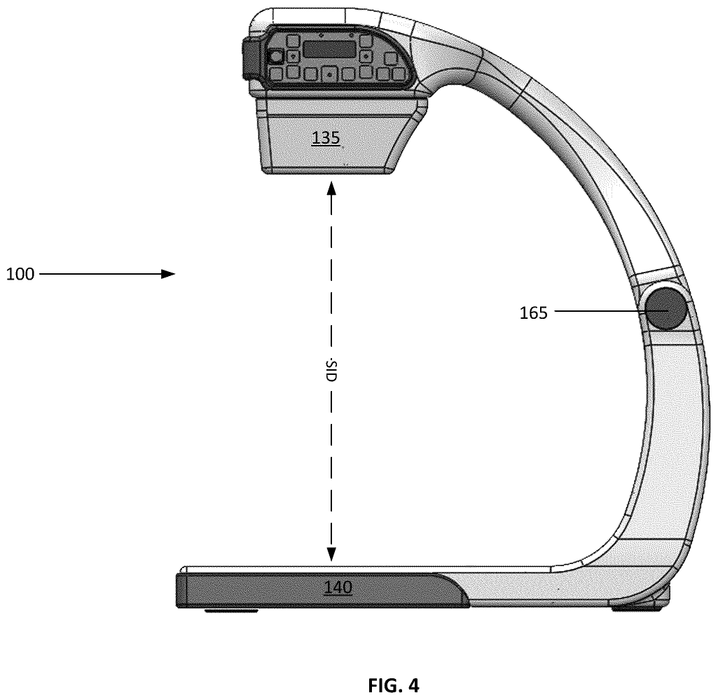

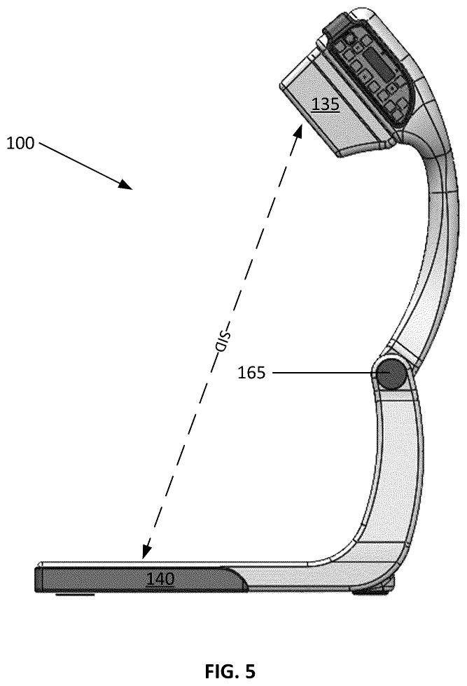

In some configurations, the X-ray source 135 can be contained in a housing that can be configured in two parts with a first part enclosing the X-ray source 135 and a second, separate part enclosing the X-ray detector 140 as shown in FIG. 2-3. In other configurations, however, the housing can be configured so that it is a single part that encloses both the X-ray source 135 and the X-ray detector 140, as shown in FIGS. 4-5.

In some configurations, the housing of the X-ray device 100 can also enclose supporting electronics 605 for the operation of the X-ray source 135, as shown in FIG. 1. The supporting electronics 605 can include a removable power source (such as a battery) and optionally an internal power supply. Thus, the power source and the power supply can be located internal to the housing of the X-ray device 100. The supporting electronics 605, as well as the supporting electronics for an image display and for wireless data upload, can also be located internal to the housing. Thus, in these configurations, the X-ray device 100 does not contain an external power cord or data cable. Incorporating the removable power source (i.e., the battery), the power supply, and the supporting electronics all within the housing allows the size and the weight of the X-ray device 100 to be reduced. Using such a configuration, the power source can easily be replaced and delivers 10,000 or even more X-ray images using just a single charge, with the exact number of images obtained with a single power source depending on the voltage and current parameters required for the images taken, with a higher power setting reducing the number of images that can be obtained. If needed, the X-ray device 100 can be configured so that it can be alternately, or additionally, charged using external power from a power cord that can be plugged into a wall outlet. In other configurations, multiple power supplies can be provided for the source, detector, and control electronics, any (or all) of which can be located either internal or external to the housing. In some configurations, the power source can be configured as described in U.S. patent application Ser. No. 15/568,708 (“the '708 application”), the entire disclosure of which is incorporated herein by reference.

In some embodiments, the X-ray device 100 can be activated by a trigger located anywhere on the X-ray device 100 that can be activated easily by the operator. As well, a separate foot switch that communicates with the X-ray device 100 using a wired or Bluetooth/wireless connection can be used. Indeed, the X-ray device 100 could be equipped with both a trigger on the device as well as a foot switch. The operation of any one of the triggers and/or the foot switch can also activate the image acquisition, compression/decompression, handling, and video/image display functions both contained within the X-ray device 100 and on the separate image display device or tablet. A remote control device can also be used to aid the repositioning of the X-ray source or detector to outside of the surgical field using a motorized system or electronically actuated release. In this manner, the medical personnel or other operator can control both the manner in which the X-ray images are obtained by holding or guiding the X-ray device 100 while also viewing the results obtained from the imaging on a conveniently located display. The ability to easily guide or manipulate the X-ray device 100 by hand, while simultaneously viewing the image results, allows the operator to obtain the X-ray images or video information needed to support the desired medical procedure similar to the way digital still and video cameras in cell phones and other personal electronic devices have enabled new and more effective ways to document, communicate, use, and share visual information. These configurations thus enable quicker and better medical decisions and encourage more frequent imaging “snapshots” during medical procedures, allowing medical practitioners to act on better information, with the result being better medical outcomes for the patient.

In FIG. 2, the X-ray device 100 has a first portion 155 that contains the X-ray source 135 and the associated supporting electronics. The X-ray device 100 also has a second portion 161 that contains the X-ray detector 140 and its associated electronics. The first portion 155 and the second portion 161 are connected using hinge (or joint) 165. The bottom of the X-ray device 100 contains an opening 175 that can be used when attaching the X-ray device 100 to an external support structure.

In some configurations, the X-ray device 100 can be connected to a stationary external (or support) structure so that it can rotate, or be positioned, around an object being analyzed, such as those described in the '708 application” and U.S. Pat. No. 10,849,579 (“the '579 patent”), the entire disclosures of which are incorporated herein by reference. Attaching the handheld or portable X-ray device 100 to a support structure allows the operator to position the portable X-ray device 100 as needed for a series of imaging procedures, while freeing medical personnel to attend to other duties. As well, it leaves the hands of the operator free for other actions. For example, during a medical procedure (including surgery), attaching the handheld or portable X-ray device 100 to a support structure allows the medical personnel to take many actions, but then easily image the patient when needed using the pre-selected positioning of the portable X-ray device 100. When the medical procedure is complete, the X-ray device 100 can be removed from the support structure and taken to another location for use or storage. In some configurations, the X-ray device 100 can be connected to the external (or support) structure using any mechanism, including those clamping mechanisms described in U.S. Pat. No. 10,674,974 (“the '974 patent”) and U.S. Pat. No. 10,856,822 (“the '822 patent”), the entire disclosures of which are incorporated herein by reference.

In other configurations, though, the handheld or portable X-ray device 100 can be connected to a movable support structure, such as those described in the '708 application and the '579 patent. In such configurations, the movable support structure can be configured to move across a floor while supporting the X-ray device 100. Thus, the movable support structure can comprise one or more wheels, shelves, handles, monitors, computers, stabilizing members, limbs, legs, struts, cables, and/or weights (to counterbalance the weight of the imaging arm and/or any other component and prevent tipping the movable support structure).

In some configurations, the X-ray device 100 and/or the external support structure can comprise any suitable locking mechanism that can selectively lock and/or unlock the rotation of the C-arm 105 around the object being analyzed. For instance, the locking mechanism can comprise a manually engaged clamp, a detent mechanism, motorized lock, electric lock, radio controlled lock, remotely engaged clamp, and/or any other suitable mechanism that can be used to lock and release the orbital rotation of the C-arm 105.

The X-ray devices 100 can also be connected to any type of electronic device with a wireless or a wired connection. In these embodiments, the X-ray device 100 itself can analyze, process, and/or transmit image or video data to the desired electronic device, (such as a desktop computer, laptop computer, tablet, cellphone, etc. . . . ), which can be used to view the X-ray images and, in some configurations, further analyze the X-ray images from the X-ray detector. In other embodiments, however, the X-ray detector 140 can be connected with any wireless communication device that can be paired with the desired electronic device, such as tablet 400 shown in FIG. 3. In the wired embodiments, the X-ray device can contain communication cables that connect the detector to the desired electronic device, such as a computer, which can be used to analyze the X-ray images from the detector.

During any X-ray imaging process, the X-ray detector operation can be coordinated with the operation of the X-ray source using, for example, a sensor or interlock system. The X-ray source and the X-ray detector should both be ready for operation when the X-ray device is activated in order to avoid the possibility of irradiating the patient with ionizing radiation when the imaging system is not ready to capture an image. If this occurs, the patient can receive the ionizing radiation with no compensating medical benefit from the desired X-ray images.

There are several ways this coordination can be implemented. In the configurations where the X-ray source and the X-ray detector are separated (and not configured into a C-arm), the coordination can be performed through wireless communication. In the configurations where the flat panel X-ray detector is operated in a continuous read-out mode and the X-ray source is continuously operated, this coordination can be accomplished using a simple “on” and “off” signal to the X-ray source and the X-ray detector simultaneously.

In the configurations that use pulsed X-ray operation, the X-ray source and the detector can be coordinated more carefully. Pulsed X-ray operation can often be used for fluoroscopy, or for stop-motion X-ray radiography, and provides benefits of higher instantaneous X-ray intensity or power to the imaging detector (for a given time-averaged X-ray intensity or total dose). Therefore, less quantum noise and a better signal-to-noise ratio in the image is produced, as well as other image quality benefits for a given total X-ray dose to the patient. The X-ray detector that is used in such a configuration will typically require some amount of time (typically measured in milliseconds or less) to readout an image before it can be ready to receive X-ray irradiation and capture a new image. Therefore, the X-ray detector 140 will typically provide a “ready” signal to tell the X-ray source to produce the X-ray radiation. In some configurations, this ready signal can be provided with hall (magnetic), optical, or mechanical sensors. In order to produce a fluoroscopic image with a significant number of sequential X-ray images per second (typically ranging from about 4 images per second up to about 30 images per second or more), the coordination between the X-ray source and the flat-panel X-ray detector can occur rapidly and repeatedly many times per second with a typical time between X-ray pulses ranging from about 100 milliseconds for a rate of about 10 frames per second, down to about 30 milliseconds for a rate of about 30 frames per second.

The X-ray device 100 can be controlled by any operator, such as a clinician, a doctor, a radiologist, a technician, or other medical professional using the input/output (I/O) mechanism. In some embodiments, the operator can control the X-ray device 100 from a central system control, such as a system control console adjacent the device. The operator can interface with the system control through a variety of optional user interfaces integrated with the I/O mechanism. In other configurations, the system control can remain separate from the I/O mechanism, such as using a separate user interface and display external to the portable X-ray device 100. The control console, the user interface, or both can be located adjacent the X-ray device 100. In other embodiments, though, the control console and/or the user interface can be located remotely, such as in an adjacent room, so as to protect the operator from unnecessary exposure to X-rays.

In some configurations, the X-ray device 100 can physically be moved from one location to the next by hand as described in the '708 application and the '579, '974, and '822 patents. The ability to move the X-ray device 100 from one location of a patient (i.e., the leg) to another location (i.e., the head), not to mention the ability to move the X-ray device 100 from one patient to another patient, makes the X-ray device 100 extremely easy and convenient for use in situations where other X-ray devices can't be used, such as sporting events, military operations, and disaster relief. Indeed, the X-ray device 100 can be configured to be portable since it is configured to be carried by hand from location to location without using wheels or a gantry. Thus, the X-ray device 100 is more portable relative to some conventional X-ray devices that contain similar features. In some embodiments, the portability of the X-ray device 100 is enhanced by reducing the weight of the X-ray device.

In the '708 application, the C-shaped support arm 105 was configured to collapse the location of the X-ray source 135 and the X-ray detector 140. This configuration allowed the C-arm 105 of the X-ray device 100 to collapse on itself, making it easy to carry and transport to a new location where it could then be restored to its “C” configuration and operated. But these collapsible C-arm X-ray devices typically had a distance between the X-ray source and X-ray detector (the source-image distance (“SID”) or free space) of about 45 cm or less, limiting the medical procedures it could be used with.

Unlike these devices described in the '708 application, though, the repositionable X-ray devices 100 described herein can be configured to reposition the X-ray source and/or X-ray detector outside of the sterile field or field in which a medical procedure will be performed without moving the rest of the X-ray device 100 or without moving any external support structure to which it is attached. In some embodiments, this repositioning of the X-ray source and/or the X-ray detector can be performed by configuring the X-ray source and/or X-ray detector to be expandable, rotatable, or slidable away from its imaging position. In some configurations, the C-arm contains a structure that is expandable since it has a mechanism that allows the X-ray source or detector to be expanded (or flipped) beyond a normal “C” arm configuration. In other configurations, the C-arm contains a structure that is rotatable since it has a mechanism that allows the X-ray source or detector to be rotated outside the normal “C” arm configuration. In yet other configurations, the C-arm contains a structure that is slidable since it contains a mechanism that allows the X-ray source or detector to be slid outside the normal “C” arm configuration. Thus, the X-ray source or X-ray detector can be moved outside of the imaging site when not being used to image the patient, but then positioned inside the imaging site when imaging is needed. These configurations allow surgeons (or other medical personnel) to operate or perform medical procedures without having to move the entire X-ray device in and out of the surgical field like some conventional X-ray devices. Instead, the rest of the X-ray device 100 can remain relatively stationary and just the X-ray source and/or X-ray detector can be moved in and out of the medical procedure field.

In some embodiments, the X-ray devices 100 can be configured to reposition the X-ray source/detector by using a joint, hinge and/or linkage or similar mechanism that allows the X-ray source (or detector) to be expanded, flipped, or opened beyond a “C” configuration. One example of these configurations is shown in FIGS. 4-5. The normal operating position of the X-ray device 100 is shown in FIG. 4 with the SID between the X-ray source 135 and detector 145 ranging from about 20 cm to about 60 cm. The expanded configuration of this X-ray device 100 is shown in FIG. 5 where the X-ray source 135 has been moved away from the position illustrated in FIG. 4. In the expanded configuration, the SID between the X-ray source 135 and detector 140 can range from about 30 cm to about 80 cm or, in some embodiments, about 50 to about 65 cm. In some configurations of a portable C-arm system having an SID of about 45 cm with a square detector size of about 15 cm, if the detector were expanded upward vertically with the bottom edge was kept aligned and the X-ray source kept with a horizontal orientation, similar to the configuration shown in FIG. 5, the SID would increase to about 53 cm. This would result in an approximate 18% increase in the SID when measured from the center of the X-ray source to the center of the X-ray detector. Thus, in these embodiments the SID has been increased by about 20% without moving the rest of the X-ray device 100. With other configurations of the X-ray device 100, the SID could be increased anywhere from about 5% to about 40%.

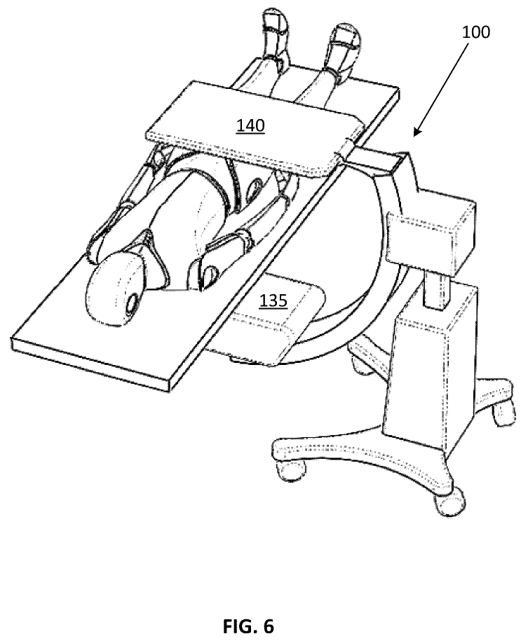

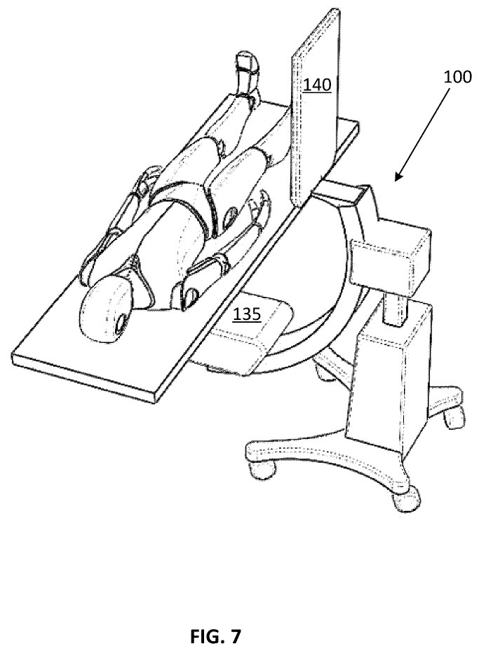

A second example of these repositionable configurations is illustrated in FIGS. 6-7 where the X-ray device 100 has been located near a table with a patient located thereon. The normal imaging position of the X-ray device 100 is shown in FIG. 6 with the SID between the X-ray source and detector ranging from about 60 to about 120 cm or, in some embodiments, about 60 cm to about 100 cm. Unlike the device shown in FIG. 4, the X-ray source 135 in FIG. 6 is located under the patient and the X-ray detector 140 is located over the patient. The expanded configuration of this X-ray device 100 is shown in FIG. 7 where the X-ray detector has been moved away from the position illustrated in FIG. 6 by using a joint, hinge, linkage, or similar mechanism to provide free space above the patient for any medical procedure to be performed. In the expanded configuration, the SID between the X-ray source and detector ranges from about 72 to about 160 cm or, in some instances, even about 80 to about 150 cm. In some configurations of a mobile C-arm system with an SID of 100 cm with a square detector size of 30 cm, if the detector were expanded upward vertically keeping the bottom edge aligned and the X-ray source was kept with a substantially horizontal orientation, similar to the position shown in FIG. 7, the SID would increase to about 116 cm. This would result in an approximate 16% increase in the SID when measured from the center of the X-ray source to center of detector. Thus, in these embodiments the SID has been increased by about 15% without moving the rest of the X-ray device 100. With other configurations of the X-ray device 100, the SID could be increased anywhere from about 10% to about 40%.

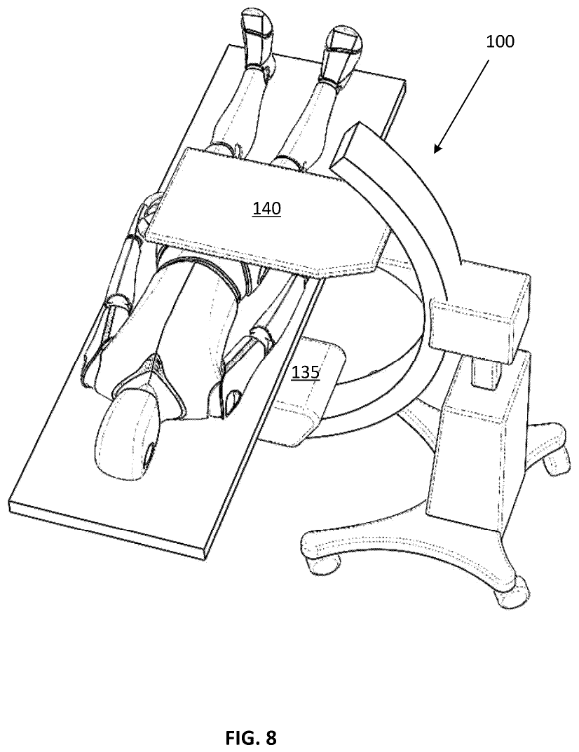

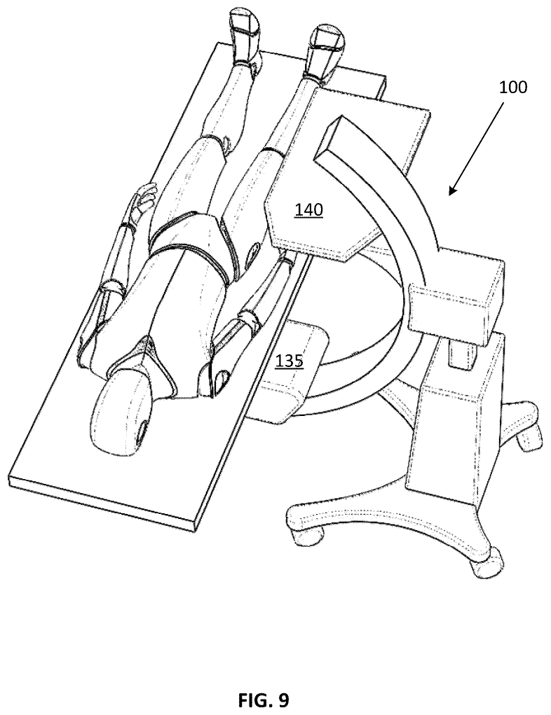

Another example of these repositionable configurations is shown in FIGS. 8-9 where the X-ray device 100 has again been located near a table with a patient located thereon. The normal imaging position of the X-ray device 100 is shown in FIG. 8 with the SID between the X-ray source 135 and X-ray detector 140 similar to that shown in FIGS. 6-7. The rotatable configuration of this X-ray device 100 is shown in FIG. 9 where the X-ray detector 140 has been rotated away from the imaging position illustrated in FIG. 8 by using a joint, hinge, linkage, or similar mechanism. In some configurations of a mobile C-arm system with a rotatable detector with an SID of 100 cm with a square detector size of 30 cm in a 30 cm×45 cm base, if the detector were rotated about 90 degrees and aligning the bottom edges of the base without moving the X-ray source, similar to the position shown in FIG. 9, the SID would increase to about 109 cm. This would result in an approximate 9% increase in the SID when measured from the center of the X-ray source to center of detector. Thus, in these embodiments the SID has been increased by about 10% without moving the rest of the X-ray device 100. With other configurations of the X-ray device 100, the SID could be increased anywhere from about 5% to about 40%. In the rotated configuration, the SID between the X-ray source and detector ranges from about 66 cm to about 160 cm or, in some instances, even about 70 cm to about 140 cm. In some configurations of a portable X-ray system with an SID of about 45 cm with a square detector size of 15 cm in a 15 cm×30 cm base, if the detector were rotated about 90 degrees and aligning the bottom edges of the base without moving the X-ray source, similar to that shown in FIG. 9, the SID would increase to about 50 cm or an approximate 10% change in SID when measured from the center of the X-ray source to the center of the X-ray detector. With other configurations, the SID could be increased anywhere from about 10% to about 40%.

In the rotated configuration, the SID between the X-ray source and detector similar to those in FIGS. 6-7 and the SID has been increased by about 5-10% without moving the rest of the X-ray device 100. This approximate SID percent change also hold true for the mobile and portable configurations with varied SID lengths and detector sizes. While the rotated configuration in FIG. 9 shows a rotation angle of about 90 degrees, it could be configured to rotate to any configuration up to about 180 degrees in either direction (e.g. clockwise or counter-clockwise), thereby increasing the SID even more.

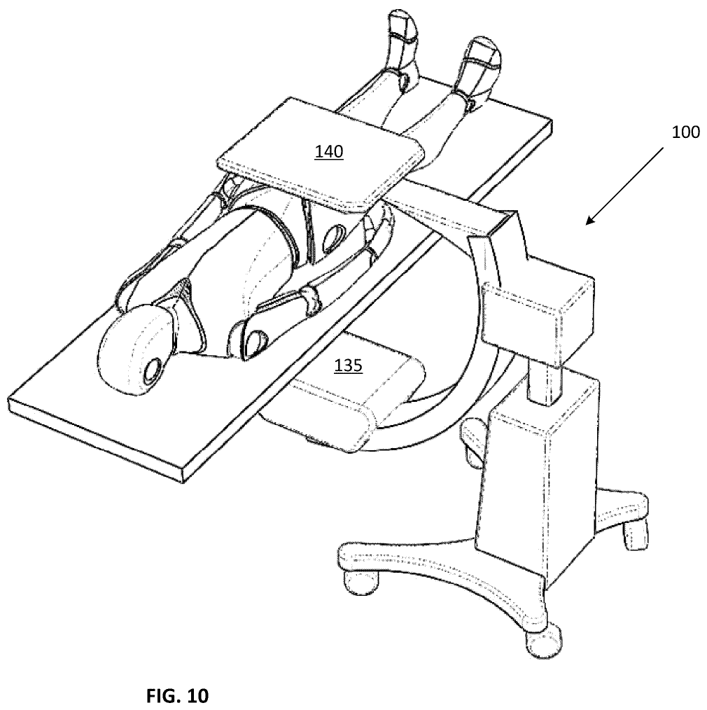

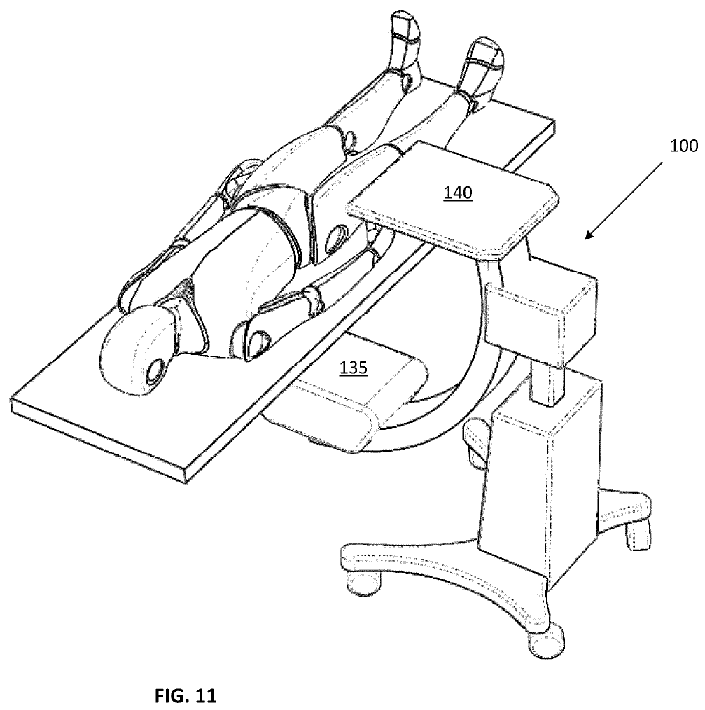

Yet another example of these repositionable configurations is shown in FIGS. 10-11 where the X-ray device 100 has again been located near a table with a patient located thereon. The normal imaging position of the X-ray device 100 is shown in FIG. 10 with the SID between the X-ray source and detector similar to those shown in FIGS. 6 and 8. The slidable configuration of this X-ray device 100 is shown in FIG. 11 where the X-ray detector 140 has been slid away from the imaging position illustrated in FIG. 10. In some configurations of a mobile C-arm system with a slidable detector with an SID of 100 cm with a square detector size of 30 cm, if the detector slid back aligning the front to the back edges without moving the X-ray source, similar to the position shown in FIG. 11, the SID would increase to about 105 cm. This would result in an approximate 4% increase in the SID when measured from the center of the X-ray source to center of detector. Thus, in these embodiments the SID has been increased by about 5% without moving the rest of the X-ray device 100. In some configurations of a portable X-ray system with an SID of 45 cm with a square detector size of 15 cm, if the detector were slid back 15 cm aligning the front and back edges without moving the X-ray source, the SID would increase to about 47 cm or an approximate 5% change in SID when measured from the center of source to center of detector. With other configurations, the SID could be increased anywhere from about 5% to about 40%. This sliding functionality can be accomplished by using linear actuators, tracked, or roller bearing mechanisms to allow the X-ray source (or detector) to slide beyond its imaging position. In some configurations, the sliding motion can be performed by an operator using either a manual and/or motorized mechanism.

The repositionable X-ray devices 100 described herein can be used to increase the free space (e.g., the opening between the X-ray source and detector or SID) between the X-ray source and X-ray detector, allowing the X-ray devices 100 to be used with a wider variety of medical procedures. The free space provides an opening for surgeons or other medical personnel to work while the C-arm 105 of the X-ray devices 100 is positioned near the patient's anatomy. A smaller free space limits the medical procedures the C-arm X-ray devices can be used with. For example, smaller free space C-arms can't be used with most arthrogram procedures that use an X-ray image or picture of the inside of a joint (e.g. shoulder, knee, wrist, ankle) after a contrast medium (sometimes referred to as a contrast agent or dye) has been injected into the joint. An arthrogram provides a clear image of the soft tissue in the joint (e.g. ligaments and cartilage) so that a more accurate diagnosis about an injury or cause of a symptom, such as joint pain or swelling, can be made. A radiologist injects the contrast medium into the joint using fluoroscopy to help guide the injection needle into the correct position. Once the injection is finished, or even during the injection, images of the joint are taken. Ninety-five percent of arthrograms performed are typically on the shoulder and hip joints which can't fit in the free space of some portable C-arm X-ray devices, including those described in the '708 application.

The repositionable X-ray devices 100 described herein have an increased free space for easier, less-hindered needle placement during arthrogram procedures. The repositionable X-ray source and/or X-ray detector of the C-arms allows the first portion of C-arm device (or head) containing the X-ray source to be positioned away from the imaging detector (or vice versa) to increase this free space. Thus, the head can pivot backwards for needle placement, then back down for fluoroscopic imaging while the contrast is injected. The repositionable X-ray devices therefore allow the X-ray detector to be positioned away from the X-ray source (or vice versa) to increase the free space of the X-ray devices 100.

This increased free space can also help in medical procedures that require the use of long tools, screws, k-wires, or needles, as well as those that use contrast to visualize vessels or joints bedside. For some conventional small C-arm X-ray devices, the free space is not large enough for the needle to be used to inject contrast. Instead, the patient needs to be moved to a shielded radiation/fluoroscopy room. But using the repositionable X-ray devices described herein, the X-ray devices 100 could be aligned to the anatomy of interest, the source and/or detector could be positioned out of the way, the contrast injected, and then the X-ray source and/or detector repositioned for fluoroscopic visualization of the vessel or joint.

The repositionable X-ray devices 100 described herein also have several other helpful features. First, they have an improved workflow that allows the medical personnel to work unhindered by the X-ray source or X-ray detector in the medical procedure field or sterile field. For example, spine surgeons will have the radiology technologist frequently move the entire C-arm X-ray device away from the patient in order to place pedicle screws or other implants into a patient and then bring the device back into the original imaging position to visualize the new screw position. This workflow is common for many types of surgery and one of the main barriers to efficient surgical workflow. Some studies suggest it can be a noticeable portion of surgery time and indicate it can take anywhere from 2-14 images to achieve the desired view during positioning for the C-arm X-ray device. It also requires technical verbal or nonverbal communication between the surgeon and radiology technologist, with mistakes and unnecessary patient exposures frequently occurring. As well, minimizing the C-arm positioning time is important to reduce radiation exposure to both the patient and medical staff. While advancements in technology (e.g., automatic C-arm positioning systems) are being explored to streamline this process, they are complex and expensive.

Another barrier to improved workflow of medical procedures is the availability of skilled radiology technologists that can effectively work in unison and communicate with the surgeon. With the repositionable X-ray devices 100 described herein, the detector (or source) can instead be merely slid, rotated, expanded, or flipped away from the operating area while locking the X-ray source (or detector) in place, thereby keeping X-ray beam alignment to the patient steady and reducing the positioning time needed for imaging. Thus, the operator (surgeon) can just push/pull the source and/or or detector back into position and maintain the anatomical alignment of the patient to the X-ray system. This functionality allows the medical personnel (surgeon) to directly control the imaging without a nurse or radiation technologist, thus reducing dependency on other medical roles (nurse, radiology technologist) and improving surgical efficiency.

The systems containing the X-ray devices described herein can also remain positioned relatively stationary near the anatomy of interest, while the X-ray source or detector is moved out of the medical procedure field. Some conventional full-size C-arms have brakes that control the position of the support arm and lock the C-arm in place. With the repositionable X-ray devices described herein, brakes are not needed. Instead, if only the X-ray source or X-ray detector is repositioned with limited degrees of freedom, it can reduce patient X-ray exposure by reducing the number of fluoroscopic images needed to realign the system to the anatomy when the patient has moved. This allows surgeons to quickly compare similar images with little movement to the C-arm positioning.

Similarly, when positioning some smaller C-arm X-ray devices, the detector is typically located under the table for the anatomical alignment required for the X-ray projection. For larger C-arms, the X-ray source can be positioned under the table on which the patient is located, with the detector above the table to reduce X-ray backscatter. Foot and ankle surgeons may place the C-arm to their side while placing the anatomy directly on the detector during the duration of the surgery and hand surgeons will often put the C-arm detector underneath the arm board and image through the board. In all of these situations, the medical professional needs to position the X-ray devices by manipulating the angle of the source and detector to the anatomy of interest. This reduces the size of the free space and, depending on the surgical site, may prevent the surgeon's ability to perform surgery. Thus, the X-ray device needs to be moved in and out the surgical site many times, creating an inefficient workflow and unrepeatable patient positioning.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation, and use may be made without departing from the principles and concepts set forth herein. Also, as used herein, the examples and embodiments, in all respects, are meant to be illustrative only and should not be construed to be limiting in any manner.

1. A repositionable X-ray device, comprising:
a C-shaped support arm;
an X-ray source contained in a first portion of the support arm near a first end of the support arm;
an X-ray detector contained in a second portion of the support arm near a second end of the support arm;
wherein the support arm contains a repositioning mechanism between the first and second portions that allows the location of the X-ray source and/or the X-ray detector to be repositioned relative to each other.

2. The X-ray device of claim 1, wherein the repositioning mechanism can be expanded to move the X-ray source or the X-ray detector outside a field of a medical procedure in which the X-ray device is used to take an image without moving the rest of the X-ray device.

3. The X-ray device of claim 1, wherein the repositioning mechanism can be rotated to move the X-ray source or the X-ray detector outside a field of a medical procedure in which the X-ray device is used to take an image without moving the rest of the X-ray device.

4. The X-ray device of claim 2, wherein the repositioning mechanism comprises a joint, hinge, or linkage.

5. The X-ray device of claim 1, wherein the repositioning mechanism can be slid to move the X-ray source or the X-ray detector outside a field of a medical procedure in which the X-ray device is used to take an image without moving the rest of the X-ray device.

6. The X-ray device of claim 5, wherein the repositioning mechanism comprises linear actuators, tracks, or a roller bearing mechanism.

7. The device of claim 1, wherein the X-ray device is configured to be portable.

8. The device of claim 1, wherein the X-ray device is configured to be mobile.

9. The device of claim 6, wherein the free space between the X-ray source and the X-ray detector has been increased by at least about 5% when the X-ray source and/or the X-ray detector has been repositioned.

10. The device of claim 9, wherein the free space between the X-ray source and the X-ray detector has been increased by up to about 40% when the X-ray source and/or the X-ray detector has been repositioned.

11. A method, comprising:
providing an X-ray device containing:
a C-shaped support arm;
an X-ray source contained in a first portion of the support arm near a first end of the support arm;
an X-ray detector contained in a second portion of the support arm near a second end of the support arm;
wherein the support arm contains a repositioning mechanism between the first and second portions that allows the location of the X-ray source and/or the X-ray detector to be repositioned relative to each other;
taking an X-ray image of a patient using the X-ray device;
repositioning the X-ray source or the X-ray detector; and
performing a medical procedure on the patient near the location where the X-ray source or the X-ray detector was previously located.

12. The method of claim 11, wherein the repositioning mechanism can be expanded to move the X-ray source or the X-ray detector outside the field of the medical procedure in which the X-ray device is used to take an image without moving the rest of the X-ray device.

13. The method of claim 11, wherein the repositioning mechanism can be rotated to move the X-ray source or the X-ray detector outside a field of the medical procedure in which the X-ray device is used to take an image without moving the rest of the X-ray device.

14. The method of claim 12, wherein the repositioning mechanism comprises a joint, hinge, or linkage.

15. The method of claim 11, wherein the repositioning mechanism can be slid to move the X-ray source or the X-ray detector outside a field of the medical procedure in which the X-ray device is used to take an image without moving the rest of the X-ray device.

16. The method of claim 15, wherein the repositioning mechanism comprises linear actuators, tracked, or roller bearing mechanism.

17. The method of claim 11, wherein the X-ray device is configured to be portable.

18. The method of claim 11, wherein the X-ray device is configured to be mobile.

19. The method of claim 16, wherein the free space between the X-ray source and the X-ray detector has been increased by at least about 5% when the X-ray source and/or the X-ray detector has been repositioned.

20. The method of claim 19, wherein the free space between the X-ray source and the X-ray detector has been increased by up to about 40% when the X-ray source and/or the X-ray detector has been repositioned.
